(19) United States
(12) Patent Application Publication
(10) Pub. No.: US 2014/0378718 A1
(43) Pub. Date: Dec. 25, 2014

(54) METHOD FOR REDUCING ACIDS IN CRUDE OIL

(71) Applicant: Baker Hughes Incorporated, Houston, TX (US)

(72) Inventors: Zhenning Gu, Sugar Land, TX (US); Jerry M. Basconi, The Woodlands, TX (US); Corina L. Sandu, Pearland, TX (US); Lawrence N. Kremer, The Woodlands, TX (US); Jerry J. Weers, Richmond, TX (US)

(73) Assignee: BAKER HUGHES INCORPORATED, Houston, TX (US)

(21) Appl. No.: 14/312,145

(22) Filed: Jun. 23, 2014

Related U.S. Application Data
(60) Provisional application No. 61/838,681, filed on Jun. 24, 2013.

(51) Int. Cl. C10G 45/30; C07C 7/12

(52) U.S. Cl. CPC C10G 45/30; C07C 7/12
USPC 585/16; 208/263; 208/14; 585/824

(57) ABSTRACT

Introducing an additive into a crude oil may result in the crude oil having comparatively lower acid levels as compared to an otherwise identical crude oil absent the additive. The additive may include nanoparticles of metal oxides, oil soluble hydrogen donors, and/or heavy amines. The oil soluble hydrogen donors may be or include 1,2,3,4-tetrahydronaphthalene; 1,2,3,4-tetrahydrdroquinoline; 9,10-dihydroanthracene; 9,10-dihydrophenanthrene; and combinations thereof. The heavy amines may be or include alkyl amines, alkanolamines, polyethylene amines, polypropylene amines, and combinations thereof.

### CROSS-REFERENCE TO RELATED APPLICATION

This application claims the benefit of Provisional Patent Application No. 61/838,681 filed Jun. 24, 2013, which is incorporated by reference herein in its entirety.

### BACKGROUND OF THE INVENTION

1. Field of the Invention

The invention relates to methods for reducing the concentration of acids in crude oil. The invention particularly relates to additive compositions useful for reducing the concentration of carboxylic acids in crude oil.

2. Background of the Art

Hydrocarbons, such as crude oil, may contain acids in several forms. These acids may be mineral acids such as hydrochloric, phosphoric, hydrogen sulfide and various oxidized forms of hydrogen sulfide such as sulfuric acid. Organic acids are also common in crude oil.

The most common form of organic acids is carboxylic acids. Such acids are characterized by a labile hydrogen associated with an oxygen which itself is adjacent to a carbonyl group. This structure is commonly shown as in the art as having a general formula R—CO₂H. While lower molecular weight carboxylic acids may be easily removed from crude oil by washing with dilute bases, higher molecular weight organic acids are not always so easily removed. Also, some carboxylic acids may be produced during refining. Finally, water washes to remove acids may, in some situations, create new problems of greater scope than the carboxylic acids being removed.

Problems caused by carboxylic acids during crude oil production and processing may include corrosion and fouling. Further, when in acid form, carboxylic acids may be easily distilled and thus be found in refined products. It may be desirable in the art of producing or refining hydrocarbons to reduce or eliminate the amount of carboxylic acids from crude and refined hydrocarbons using an additive. Reducing the concentration of carboxylic acids in a crude oil reservoir would bring the maximum benefits in terms of eliminating the corrosion and fouling issues caused by the acidic species from early stages of crude oil production, transportation, storage and processing.

### SUMMARY OF THE INVENTION

There is provided, in one form, a method for at least partially decreasing an amount of acids in crude oil by introducing an effective amount of oil-soluble hydrogen donors and an effective amount of calcined metal oxide nanoparticles into a crude oil to decrease the acids as compared to an otherwise identical crude oil absent the oil-soluble hydrogen donors and calcined metal oxide nanoparticles. The calcined metal oxide nanoparticles may be introduced into the crude oil at the same time or different time from the oil-soluble hydrogen donors.

There is further provided in another non-limiting embodiment of the method for at least partially decreasing an amount of acids in crude oil by introducing an effective amount of heavy amines and an effective amount of metal oxide nanoparticles into a crude oil to decrease the acids as compared to an otherwise identical crude oil absent the heavy amines and metal oxide nanoparticles. The metal oxide nanoparticles are introduced into the crude oil at the same time or different time from the heavy amines.

In another non-limiting embodiment, a treated crude oil composition is described. The treated crude oil composition may include a crude oil comprising acids, metal oxide nanoparticles in an amount ranging from about 5 wt % to about 40 wt %, and heavy amines in an amount ranging from about 5 wt % to about 40 wt %. The treated crude oil composition may include a decreased amount of acids as compared to an otherwise identical crude oil absent the metal oxide nanoparticles and the heavy amines.

In another non-limiting embodiment, a treated crude oil composition may include a crude oil comprising acids, calcined metal oxide nanoparticles in an amount ranging from about 5 wt % to about 40 wt %, and oil-soluble hydrogen donors in an amount ranging from about 5 wt % to about 40 wt %. The treated crude oil composition may include a decreased amount of acids as compared to an otherwise identical crude oil absent the metal oxide nanoparticles and the oil-soluble hydrogen donors.

### DETAILED DESCRIPTION

In one aspect, the invention is a method for reducing the concentration of carboxylic acids in crude oil within a crude oil reservoir. The method comprises introducing downhole an additive comprising nanoparticles of metal oxides, an oil soluble hydrogen donor, a heavy amine, and combinations thereof. In a non-limiting embodiment, the additive may not include an oil-soluble hydrogen donor. Alternatively, the additive may not include the heavy amine. The heavy amine may have a molecular weight greater than about 150 grams/mole in a non-limiting embodiment, or a molecular weight greater than about 180 grams/mole. In a non-limiting embodiment, the heavy amine may be a mixture of linear, branched, and cyclic ethyleneamines having a molecular weight typically above 150 grams/mole or 180 grams/mole.

Complete removal or inactivation of the carboxylic acids from the crude oil is desirable, but it should be appreciated that complete removal is not necessary for the methods discussed herein to be considered effective. Success is obtained if more of the carboxylic acids are removed or inactivated using the metal oxide nanoparticles, oil-soluble hydrogen donors, and/or heavy amines than in the absence of such components. Alternatively, the methods described are considered successful if the carboxylic acids are reduced in concentration by at least 50%. ‘Effective amount’ is defined herein to mean any amount of a component introduced into the crude oil that decreases the concentration of, removes, or otherwise inactivates the acids within the crude oil as compared to an otherwise identical crude oil absent the component(s).

In a non-limiting embodiment, the additive may be introduced into crude oil as it is being produced or even after it leaves the well, such as during the refining of the crude oil. Alternatively, the additive may be introduced at the surface, e.g. a produced crude oil, a refinery crude oil, and the like. A ‘produced crude oil’ is defined herein to be any produced crude oil that has not been further treated or refined. A ‘refinery crude oil’ refers to a crude oil that is currently being treated or refined or one that has already been treated or refined. ‘Treated and ‘refined’ with reference to the crude oil is defined as further processing or treatment of the crude oil.

In a non-limiting embodiment, the crude oil may be a bio-derived crude oil, such as but not limited to an algae feedstock. Hydrothermal liquefaction (HTL) of biomass provides a direct pathway for liquid biocrude production as described in more detail in “Process Development for Hydrothermal Liquefaction of Algae Feedstocks in a Continuous-Flow Reactor”, Douglas C. Elliott, et al., Algal Research 2 (2013) 445-454, which is incorporated by reference herein in its entirety. Wet algae slurries may be converted into a bio-derived crude oil by HTL. Hydrothermal processing uses water-based slurries with sufficient temperature and pressure to maintain the water in the liquid phase. The TAN for bio-derived crude oils may be as high as 74, which makes processing of such crudes undesirable.

The additive may be introduced into the water-based slurry feed prior to entering an HTL unit, introduced into the bio-derived crude oil feed from the HTL unit, and combinations thereof where the acids therein may be catalytically decomposed into alkanes and carbon dioxide. The additive may include the metal oxides, the oil soluble hydrogen donor, and/or the heavy amine, and combinations thereof. In a non-limiting embodiment, the additive may not include an oil-soluble hydrogen donor. Alternatively, the additive may not include the heavy amine.

In a non-limiting embodiment, a method for hydrothermally processing biomass may include contacting a biomass feed, such as an algae-based feed in a non-limiting embodiment, with water in the presence of the additive under effective hydrothermal processing conditions to produce a multi-phase product. The multi-phase product may be separated to produce at least a gas phase portion, a liquid hydrocarbon product, an aqueous portion, and a solids portion. The liquid hydrocarbon product may be separated from the multi-phase product where at least a portion of the liquid hydrocarbon product may be further processed (e.g. hydroprocessed). Alternatively, the additive may contact the liquid hydrocarbon product, in lieu of or in addition to first contacting the biomass feed, to decrease the TAN of the liquid hydrocarbon product, i.e. the bio-derived crude oil.

In a non-limiting embodiment, the effective hydrothermal processing conditions may include a temperature ranging from about 150 C independently to about 500 C, and a pressure ranging from about 450 kPag to about 30 Mpag.

When introduced at the surface, it may be necessary to further heat the crude oil including the additive. For example, in one embodiment, the additive may be introduced into the crude oil within a heat treater. If necessary, it may be desirable to further heat the crude oil to a temperature of 150 C or higher in a non-limiting embodiment, alternatively the temperature may range from about 100 C independently to about 300 C or higher, alternatively from about 150 C independently to about 290 C, or from about 200 C independently to about 600 C. At lower temperatures, such as 150 C, the conversion of the acids may take longer periods of time than if higher temperatures were used.

A toping unit is another surface treatment device that may heat the crude oil and the additive to a temperature in excess of 150 C, alternatively from about alternatively the temperature may range from about 100 C independently to about 300 C or higher, alternatively from about 150 C independently to about 290 C, or from about 200 C independently to about 400 C. In such a unit, the concentration of water is typically very low which may, in some embodiments, be advantageous to the use of the additive.

When introducing or contacting the crude oil with the additive at the surface, the process may occur at atmospheric temperature, but elevated pressures may be undesirable during such a process. While not wishing to be bound by any theory, it is nevertheless believed that increased pressure of CO₂ (one of the primary gases downhole) may limit the reaction by Le Chatelier's principal. Operating at atmospheric pressure and/or using other gases, such as nitrogen, avoids this issue. Le Chatelier's principal describes a chemical system at equilibrium, which when experiencing a change, may shift the equilibrium to counteract the imposed change by altering concentrations of components, temperature, volume, and/or pressure.

The metal oxides useful with the method of the invention may be one or more selected from the group consisting of alkaline earth metal oxides, transition metal oxides, and post-transition metal oxides. For the purposes of the application, the term “post-transition metal” is meant one or more of aluminum, gallium, indium, tin, thallium, lead and bismuth. In another non-limiting embodiment herein, the nano-sized particles are oxides and hydroxides of elements of Groups IA, IIA, IVA, IIB and IIIB of the previous IUPAC American Group notation. These elements include, but are not necessarily limited to Mg, Ca, Ti, Zn and/or Al.

The nanoparticles useful with the method of the application may be prepared using any method known to be useful to those of ordinary skill in the art. For example, in one such embodiment, the nanoparticles may be prepared using a controlled precipitation method. In another embodiment, the nanoparticles may be prepared using controlled pyrolysis reactions.

In a non-limiting embodiment, the metal oxide nanoparticles may be stabilized in a hydrocarbon carrier in an overbase form, or in a hydrocarbon mixture that includes particle-dispersing polymers. Alternatively, the metal oxide nanoparticles may be generated in situ under the HTL conditions by adding a metal hydroxide slurry (e.g. a magnesium hydroxide slurry) to the HTL feed prior to the feed entering the reactor. In yet another non-limiting embodiment, the metal oxide nanoparticles may be porous nanoparticles having larger surface areas, such as impregnated metal oxide membranes that may be used as columns. The columns may allow for increased surface area, a faster reaction, and/or the ability to regenerate the metal oxide nanoparticles in situ.

In still another embodiment, the precursor nanoparticles may be further processed by controlled calcining, which may result in a phase transition or removal of volatile residues at the surface of the nanoparticles. In a non-limiting instance, calcination may enhance surface reactivity of the metal oxide nanoparticles. Calcination of the nanoparticles is a thermal treatment process to the nanoparticles where the nanoparticles are heated at a temperature ranging from about 300 C independently to about 1400 C, or from about 600 C independently to about 1000 C. The heating may occur for a period of time ranging from about 2 hours independently to about 7 days, alternatively from about 5 hours independently to about 48 hours, or from about 10 hours independently to about 24 hours.

In another non-limiting embodiment, the nanoparticles may be overbased nanoparticles. In a non-limiting embodiment, one method of preparing overbased metal oxide nanoparticles may include forming a mixture of a base with the desired metal, a complexing agent, and a non-volatile diluent. In a non-limiting example, the mixture of the base may include Mg(OH)₂; the complexing agent may include a fatty acid, such as a tall oil fatty acid that is present in a quantity much less than that required to stoichiometrically react with the base of the desired metal. The mixture may be heated to a temperature ranging from about 250° C. to about 350° C. to produce the overbased metal oxide nanoparticles.

In a non-limiting embodiment, the nanoparticles may be introduced into the crude oil in an amount ranging from about 5 wt % independently to about 40 wt %, or from about 2 wt % independently to about 25 wt %. The nanoparticles may have a mean particle size ranging from about 5 μm independently to about 100 nm. In some embodiments, the nanoparticles may have a mean particle size of from about 1 independently to about 90 nm. In still other embodiments, the mean particle size may be from about 20 independently to about 75 nm.

The oil-soluble hydrogen donor may be or include, but is not limited to, 1,2,3,4-tetrahydronaphthalene; 1,2,3,4-tetrahydrdroquinoline; 9,10-dihydroanthracene; 9,10-dihydrophenanthrene; and combinations thereof. Any compound known to function as a hydrogen donor in a hydrocarbon may be used.

The nanoparticles of metal hydroxides may be combined with the oil-soluble hydrogen donors to prepare the additive prior to its introduction downhole. The additives are prepared using a weight ratio of nanoparticles to hydrogen donors ranging from about 1:99 independently to about 99:1. In some embodiments, the ratio is from about 1:19 independently to about 19:1, and in still other embodiments, the ratio is from about 1:9 independently to about 9:1. In at least one embodiment, the ratio is from about 1:3 independently to 3:1. In an alternative non-limiting embodiment, the components of the additive are not admixed prior to introducing them downhole. Alternatively, the effective amount of the oil-soluble hydrogen donors may range from about 5 wt % independently to about 40 wt % based on the total amount of the crude oil to be treated, or from about 10 wt % independently to about 20% in another non-limiting embodiment.

The heavy amines may be or include, but are not limited to, tetraethylenepentamine, alkyl amines, alkanolamines, polyethylene amines, polypropylene amines, and combinations thereof. The weight ratio of nanoparticles to heavy amines may range from about 0.01:2 independently to about 1:1. In some embodiments, the ratio is from about 0.1:1.5 independently to about 0.5:1. Alternatively, the effective amount of the heavy amines may range from about 5 wt % independently to about 40 wt % based on the total amount of the crude oil to be treated, or from about 10 wt % independently to about 40 wt % in another non-limiting embodiment.

The carboxylic acids may be irreversibly converted into non-acidic species. The additive may be introduced downhole into the reservoir holding crude oil. The additive may be introduced downhole using any method known to be useful to those of ordinary skill in the art. For example, it may be introduced via any subsurface injection point using pumps, hydraulic pressures or even gas pressure. For example, it may be introduced using nitrogen, carbon dioxide, or even steam in some embodiments.

One method of measuring acidity within a crude oil is the total acid number (TAN), which is reported as mg KOH/g sample. In one embodiment, the additive may be introduced into a crude oil having a TAN ranging from about 0.1 independently to about 20. In another embodiment, the TAN may range from about 1 independently to about 10. In still another embodiment, the TAN may range from about 2 independently to about 5.

When introducing the additive into the crude oil, the additive may be used at a temperature that is greater than ambient, which may increase the speed of converting the acids. In one non-limiting embodiment, the additive may be introduced into a crude oil reservoir having a temperature of at least 20° C., alternatively from about 25° C. independently to about 350° C. or higher, or from about 30° C. independently to about 150° C. in another non-limiting embodiment.

The total feed rate or total dosage of the additive may generally be determined by the original TAN and target TAN of the crude oil in a reservoir, as well as by the specific operating parameters of the crude oil production units. Those of ordinary skill in the art in operating such a unit will know how to make such determinations based upon the above conditions. Nevertheless, in some non-limiting embodiments, the feed rate of the additives may be from about 100 independently to 10,000 ppm by weight of the additive in the recoverable crude oil in the reservoir. ‘Original TAN’ is defined as the TAN of the crude oil prior to the introduction of the oil-soluble hydrogen donors and/or heavy amines. ‘Target TAN’ is defined as the desired TAN of the crude oil.

The additive of the application may be present at a material concentration, namely a concentration that is sufficient to reduce the acidity of the crude oil in a reservoir by at least 10 percent based upon the TAN in a non-limiting embodiment. In other non-limiting embodiments, the material concentration of the additive may be sufficient to reduce the acidity of a hydrocarbon (e.g. crude oil) by at least 25 percent based upon total acid number. In still other non-limiting embodiments, the material concentration of the additive is sufficient to reduce the acidity of a hydrocarbon (e.g. crude oil) by at least 50 percent based upon total acid number.

The invention will be further described with respect to the following Examples, which are not meant to limit the invention, but rather to further illustrate the various embodiments.

### EXAMPLES

The following examples are provided to illustrate the invention. The examples are not intended to limit the scope of the invention and they should not be so interpreted. Amounts are in weight parts or weight percentages, unless otherwise indicated.

### Example 1

A simulated acidic oil sample was prepared by adding a sufficient amount of technical grade naphthenic acid (Aldrich) to a heavy mineral oil (Aldrich) to reach a TAN of 7.85 (as determined by ASTM testing method D-664). Such sample was divided into three equal portions. One portion was set as a blank. The second portion was treated with about 1000 ppm of MgO nanoparticles dispersed in aromatic solvents, such as a SOLVESSO™ 150 distributed by EXXON MOBIL™, which is an aromatic solvent having a flash point of 150° F. The third portion was treated with about 1000 ppm of calcined MgO nanoparticles dispersed in aromatic solvents, such as ‘150 solvent’. The three oil samples were each heated in a resin reaction kettle (with condenser attached) at 288° C. for 5 hours with constant stirring, under continuous purge of a simulated sour gas (1% hydrogen sulfide in nitrogen). After heating, the three oil samples were tested for TAN using the ASTM method specified above. Example 1 shows that the additives based on alkaline earth metal oxide nanoparticles were able to reduce the acidity of oil and the calcined alkaline earth metal oxide nanoparticles showed higher efficiency on acidity reduction. As noted in TABLE 1, calcined nanoparticles decrease the TAN more than non-calcined nanoparticles.

The results are listed in Table-1.

TABLE 1

| Sample | TAN after heating |
|---|---|
| Blank | 7.00 |
| 2^{nd} Portion | 4.32 |
| 3^{rd} Portion | 3.83 |

### Example 2

A simulated acidic oil sample was prepared the same as described in Example 1 with an initial TAN of 4.22. This sample was divided into two equal portions. One portion was set as a blank. The second portion was treated with an oil soluble additive formula which contains an overbase stabilized MgO dispersion in an amount of 10.92 g, 1,2,3,4-tetrahydroquinoline in an amount of 2.76 g, and tetraethylenepetamine in an amount of 1.48 g; the approximate ratio of the overbased stabilized MgO to 2,3,4-tetrahydroquinoline was 8:2:1. The oil samples were each heated in the same apparatus as in Example 1 at 288° C. for 4 hours with constant stirring, under a continuous nitrogen purge. Example 2 shows that the combination of MgO, 1,2,3,4-tetrahydroquinoline, and tetraethylenepentamine effectively reduce TAN of acidic oil at the elevated temperature.

The TAN results of the oil samples after heating are listed in Table-2.

TABLE 2

| Sample | TAN after heating |
|---|---|
| Blank | 3.96 |
| Treated | 1.91 |

### Example 3

A simulated acidic oil sample was prepared the same as that in Example 1 with an initial TAN of 5.38. This sample was divided into four equal portions. The first portion was used as a blank and did not have anything added thereto. The second portion was treated with 1.4% of a magnesium overbase liquid. The third portion was treated with 0.1% of tetraethylenepentamine. The forth portion was treated with 0.76% of the magnesium overbase liquid and 0.05% of tetraethylenepentamine. The samples were each heated in the same apparatus as in Example 1 at 288° C. for 4 hours with constant stirring, under a continuous nitrogen purge. As noted in TABLE 3, the use of the magnesium overbase liquid and the tetraethylenepentamine have synergistic results when used together as compared to when each is used alone.

The TAN results of the samples after heating are listed in Table-3.

TABLE 3

| | TAN after heating, |
|---|---|
| Sample | mg KOH/g |
| Blank | 5.02 |
| 2^{nd} Portion | 4.07 |
| 3^{rd} Portion | 3.11 |
| 4^{th} Portion | 1.98 |

In the foregoing specification, the invention has been described with reference to specific embodiments thereof, and has been described as effective in providing methods and compositions for decreasing an acid content with crude oils. However, it will be evident that various modifications and changes can be made thereto without departing from the broader spirit or scope of the invention as set forth in the appended claims. Accordingly, the specification is to be regarded in an illustrative rather than a restrictive sense. For example, specific crude oils, oil-soluble hydrogen donors, heavy amines, and nanoparticles, falling within the claimed parameters, but not specifically identified or tried in a particular composition or method, are expected to be within the scope of this invention.

The present invention may suitably comprise, consist or consist essentially of the elements disclosed and may be practiced in the absence of an element not disclosed. For instance, a method may consist of or consist essentially at least partially decreasing an amount of acids in crude oil by introducing an effective amount of oil-soluble hydrogen donors and an effective amount of calcined metal oxide nanoparticles into the crude oil to decrease the amount of acids as compared to an otherwise identical crude oil absent the oil-soluble hydrogen donors and calcined metal oxide nanoparticles; the crude oil comprises acids; and the calcined metal oxide nanoparticles is introduced into the crude oil at the same time or different time from the oil-soluble hydrogen donors.

Another embodiment of the method may consist of or consist essentially of at least partially decreasing an amount of acids in a crude oil by introducing an effective amount of heavy amines and an effective amount of metal oxide nanoparticles into a crude oil to decrease the acids as compared to an otherwise identical crude oil absent the heavy amines and metal oxide nanoparticles; the crude oil comprises acids; and the metal oxide nanoparticles are introduced into the crude oil at the same time or different time from the heavy amines.

The treated crude oil composition may consist of or consist essentially of a crude oil, metal oxide nanoparticles in an amount ranging from about 5 wt % to about 40 wt %, and heavy amines in an amount ranging from about 5 wt % to about 40 wt %; the treated crude oil composition may have a decreased amount of acids as compared to an otherwise identical crude oil absent the metal oxide nanoparticles and the heavy amines.

In another embodiment, the treated crude oil composition may consist of or consist essentially of a crude oil, calcined metal oxide nanoparticles in an amount ranging from about 5 wt % to about 40 wt %, and oil-soluble hydrogen donors in an amount ranging from about 5 wt % to about 40 wt %; and the treated crude oil composition may have a decreased amount of acids as compared to an otherwise identical crude oil absent the metal oxide nanoparticles and the oil-soluble hydrogen donors.

The words “comprising” and “comprises” as used throughout the claims, are to be interpreted to mean “including but not limited to” and “includes but not limited to”, respectively.

What is claimed is:

1. A method for at least partially decreasing an amount of acids in crude oil, wherein the method comprises:
introducing an effective amount of oil-soluble hydrogen donors and an effective amount of calcined metal oxide nanoparticles into a crude oil to decrease the acids as compared to an otherwise identical crude oil absent the oil-soluble hydrogen donors and calcined metal oxide nanoparticles; wherein the crude oil comprises acids; and wherein the calcined metal oxide nanoparticles are introduced into the crude oil at the same time or different time from the oil-soluble hydrogen donors.

2. The method of claim 1, wherein the crude oil is selected from the group consisting of a downhole crude oil, a produced crude oil, a refinery crude oil, a bio-derived crude oil, and combinations thereof.

3. The method of claim 1, wherein a ratio of the oil-soluble hydrogen donors to the metal oxide nanoparticles ranges from about 1:99 to about 99:1.

4. The method of claim 1, wherein the effective amount of the oil-soluble hydrogen donors ranges from about 5 wt % to about 40 wt %.

5. The method of claim 1 further comprising introducing an effective amount of heavy amines into the crude oil at the same time or different time from the calcined metal oxide nanoparticles and/or the oil-soluble hydrogen donors, to decrease the acids.

6. The method of claim 5, wherein a ratio of the heavy amines to the calcined metal oxide nanoparticles ranges from about 2:0.01 to about 1:1.

7. The method of claim 1, wherein the effective amount of the calcined metal oxide nanoparticles ranges from about 5 wt % to about 40 wt %.

8. The method of claim 1, wherein the calcined metal oxide nanoparticles are overbased calcined metal oxide nanoparticles.

9. The method of claim 1 where the oil-soluble hydrogen donors are selected from the group consisting of 1,2,3,4-tetrahydronaphthalene; 1,2,3,4-tetrahydrdroquinoline; 9,10-dihydroanthracene; 9,10-dihydrophenanthrene; and combinations thereof.

10. A method for at least partially decreasing an amount of acids in crude oil, wherein the method comprises:
introducing an effective amount of heavy amines and an effective amount of metal oxide nanoparticles into a crude oil to decrease the acids as compared to an otherwise identical crude oil absent the heavy amines and metal oxide nanoparticles; wherein the crude oil comprises acids; and wherein the metal oxide nanoparticles are introduced into the crude oil at the same time or different time from the heavy amines.

11. The method of claim 10, wherein the crude oil is selected from the group consisting of a downhole crude oil, a produced crude oil, a refinery crude oil, a bio-derived crude oil, and combinations thereof.

12. The method of claim 10, wherein a ratio of the heavy amines to the metal oxide nanoparticles ranges from about 2:0.01 to about 1:1.

13. The method of claim 10, wherein the effective amount of the heavy amines ranges from about 5 wt % to about 40 wt %.

14. The method of claim 10, wherein the metal oxide nanoparticles are calcined metal oxide nanoparticles.

15. The method of claim 10, wherein the metal oxide nanoparticles are overbased metal oxide nanoparticles.

16. A treated crude oil composition comprising:
a crude oil comprising acids;
metal oxide nanoparticles in an amount ranging from about 5 wt % to about 40 wt %;
heavy amines in an amount ranging from about 5 wt % to about 40 wt %; and
wherein the treated crude oil composition comprises a decreased amount of acids as compared to an otherwise identical crude oil absent the metal oxide nanoparticles and the heavy amines.

17. The treated crude oil composition of claim 15, wherein the crude oil is selected from the group consisting of a downhole crude oil, a produced crude oil, a refinery crude oil, a bio-derived crude oil, and combinations thereof.

18. The treated crude oil composition of claim 15, wherein the metal oxide nanoparticles are overbased metal oxide nanoparticles.

19. The treated crude oil composition of claim 15, wherein the metal oxide nanoparticles are calcined metal oxide nanoparticles.

20. A treated crude oil composition comprising:
a crude oil comprising acids;
calcined metal oxide nanoparticles in an amount ranging from about 5 wt % to about 40 wt %;
oil-soluble hydrogen donors in an amount ranging from about 5 wt % to about 40 wt %; and
wherein the treated crude oil composition comprises a decreased amount of acids as compared to an otherwise identical crude oil absent the metal oxide nanoparticles and the oil-soluble hydrogen donors.

21. A method for at least partially decreasing an amount of acids in a biomass feed, wherein the method comprises:
contacting a biomass feed with water in the presence of an additive under effective hydrothermal processing conditions to produce a multi-phase product; wherein the biomass feed comprises acids; wherein the additive comprises an effective amount of metal oxide nanoparticles to decrease the acids as compared to an otherwise identical biomass feed absent the metal oxide nanoparticles; and
at least partially decreasing the amount of acids in the biomass feed.

22. The method of claim 21, further comprising separating the multi-phase product into at least two portions selected from the group consisting of a gas phase portion, a liquid hydrocarbon product, an aqueous portion, a solids portion, and combinations thereof.

23. The method of claim 21, wherein the additive further comprises at least one additional component selected from the group consisting of an oil-soluble hydrogen donor, a heavy amine, and combinations thereof.

24. The method of claim 21, wherein the metal oxide nanoparticles are calcined metal oxide nanoparticles.

25. A treated bio-derived water-based slurry composition comprising:
a bio-derived water-based slurry comprising acids;
metal oxide nanoparticles in an amount ranging from about 5 wt % to about 40 wt %;
wherein the treated bio-derived water-based slurry comprises a decreased amount of acids as compared to an otherwise identical bio-derived water-based slurry absent the metal oxide nanoparticles.

26. The treated bio-derived water-based slurry composition of claim 25, wherein the additive further comprises at least one additional component selected from the group consisting of an oil-soluble hydrogen donor, a heavy amine, and combinations thereof.

27. The treated bio-derived water-based slurry composition of claim 25, wherein the metal oxide nanoparticles are calcined metal oxide nanoparticles.
